(19) United States
(12) Patent Application Publication
(10) Pub. No.: US 2005/0245541 A1
(43) Pub. Date: Nov. 3, 2005

(54) Methods for treating alcoholism

(76) Inventors: Elliot Ehrich, Lincoln, MA (US); Trevor Mundel, Carlisle, MA (US)

Correspondence Address: ELMORE CRAIG & VANSTONE, P.C., 209 MAIN STREET, N. CHELMSFORD, MA, 01863, US

(21) Appl. No.: 11/082,534

(22) Filed: Mar. 17, 2005

Related U.S. Application Data
(60) Provisional application No. 60/554,785, filed on Mar. 19, 2004.
(60) Provisional application No. 60/562,356, filed on Apr. 15, 2004.

(51) Int. Cl. A61K 31/495; A61K 31/485; A61K 31/445

(52) U.S. Cl. 514/255.04; 514/282; 514/317

(57) ABSTRACT

In the treatment of alcoholism, co-treatment with an active agent capable of offsetting unwanted adverse clinical manifestations to the treatment itself, for example, negative drug adverse clinical manifestations, greatly increases patient compliance. Increasing patient compliance, in turn, creates a better success rate and decreased recidivism or relapse. The current invention combines the use of an opioid antagonist with at least one dopamine D2 partial agonist for the treatment of alcoholism. Suitable dopamine D2 partial agonists are aripiprazole and (−)3-(3-hydroxyphenyl)-N-n-propylpiperidine [(−)-3PPP].

### RELATED APPLICATIONS

This application claims the benefit of U.S. Provisional Application No. 60/554,785, filed on Mar. 19, 2004, and U.S. Provisional Application No. 60/562,356, filed on Apr. 15, 2004. The entire contents of both provisional applications are hereby incorporated by reference.

### BACKGROUND OF THE INVENTION

Alcohol dependence is a chronic disorder that results from a variety of genetic, psychological and environmental factors. Treatment has consisted of two phases: detoxification and rehabilitation. Detoxification ameliorates the symptoms and signs of withdrawal; rehabilitation helps the patient avoid future problems with alcohol. In the past, most rehabilitative treatments have been psychosocial. With advances in neurobiology, there is increasing interest in drug therapy for alcohol dependence. For a discussion of the development of this field, see Swift, R., Drug Therapy for Alcohol Dependence, NEJM, May 13, 1999, 1482-1490. Yet, the successful treatment of alcoholism has many serious challenges and complications. For example, alcohol abuse followed by withdrawal is one of the most common causes of seizures in adults. The seizures are serious medical conditions which require more intensive treatment, usually under emergency conditions.

Others have attempted treating alcoholism following a period of abstinence by the patient, for example, by administering opioid antagonists. Opioid antagonists act by blocking the reinforcing effect of alcohol which gives rise to craving. For the treatment of alcoholism, opioid antagonists act by blocking the positive effects of alcohol which results from the release of endogenous opioids upon the consumption of alcohol. However, during the period of abstinence, symptoms of withdrawal may appear.

Thus, a treatment is needed to counteract the different negative aspects associated with treatment of alcohol dependence, in this case, withdrawal symptoms and craving. The occurrence of either of these symptoms is difficult but the combination of these two negative aspects often present insurmountable challenges to patients, even highly motivated patients. Therefore, a need exists for treatment of alcoholism tailored to decrease alcohol dependence, minimize withdrawal symptoms, especially protracted or persistent abstinence syndrome (PAS), and inhibit cravings.

Still further, often the treatment itself is perceived by the patient as “worse than the cure.” For example, once the initial detoxification begins and the patient decreases or ceases alcohol intake as per the treatment regime, the patient will often view the adverse clinical manifestations of the medication itself as unpleasant and unwanted. Then, the patient will stop taking the drug. The lack of patient compliance with the entire treatment regime is an enormous problem and accounts for a high rate of incomplete treatment and relapse. Accordingly, there is a need for counteracting the negative aspects of the drug treatment itself so as to increase compliance with the treatment regime.

### SUMMARY OF THE INVENTION

The invention is based upon the discovery that a continuity of treatment and anticipation of the timing, severity and combination of withdrawal symptoms and cravings is a key to successful treatment of alcohol dependence. The invention is also based upon the discovery that co-treatment with an active agent capable of offsetting unwanted adverse clinical manifestations to the treatment itself, for example, negative drug side effects, greatly increases patient compliance. Increasing patient compliance, in turn, creates a better success rate and decreased recidivism or relapse. The current invention is a method for treating alcoholism comprising administering to a patient a therapeutically effective amount of a combination of (i) at least one opioid antagonist (such as naltrexone, naloxone and nalmefene); and (ii) at least one dopamine D2 partial agonist in the treatment of alcoholism, including the treatment of alcohol dependence, withdrawal symptoms, PAS and cravings. The combination also reduces the neuronal excitability associated with withdrawal.

In another aspect the invention reduces the negative adverse clinical manifestations of the at least one opioid antagonist.

In another aspect, the invention improves patient compliance when treating the patient for alcoholism.

In one aspect, the factor of non-compliance is greatly reduced, preferably removed, as a contributing factor to the failure rate of treatment for alcoholism.

In yet another aspect, the at least one opioid antagonist is selected from the group consisting of naltrexone, naloxone and nalmefene.

In still another aspect, the at least one opioid antagonist is in a form selected from the group consisting of a polymorph, solvate, hydrate, dehydrate, co-crystal, anhydrous form and amorphous form or combinations thereof.

In yet a further aspect, the invention is a kit comprising at least one treatment dose of therapeutically effective amount of:
(i) at least one opioid antagonist; and
(ii) at least one dopamine D2 partial agonist
   wherein the antagonist of (i) and the dopamine D2 partial agonist of (ii) comprise a single pharmaceutical composition.

In still another aspect, the invention is a kit comprising at least one treatment dose of therapeutically effective amount of:
(i) at least one opioid antagonist; and
(ii) at least one dopamine D2 partial agonist
   wherein the antagonist of (i) and dopamine D2 partial agonist of (ii) comprise more than one pharmaceutical composition.

The invention further relates to a method for treating alcoholism by administering a pharmaceutical composition comprising (i) at least one opioid antagonist; and (ii) at least one dopamine D2 partial agonist, for a time period (a) beginning with discontinuation or reduction of alcohol intake throughout complete withdrawal, (b) beginning with discontinuation or reduction of alcohol intake until the symptoms of PAS abate, (c) during a drinking reduction program and/or (d) before (in anticipation of) or concurrently with life events that would increase the risk of relapse. In one embodiment, situational treatment is begun before treatment or resumed after treatment has ceased.

### DESCRIPTION OF THE DRAWINGS

FIG. 1A is a bar graph showing naltrexone dose response in a rodent model of alcohol administration. Naltrexone (0-6.0 mg/kg, sc) was administered to trained rat to measure its effect on ethanol drinking using an operant self-administration procedure (pressing of a lever). A dose-dependent decrease in the number of lever presses is observed with an ED₅₀ of 0.1-0.5 mg/kg.

FIG. 1B is a bar graph showing the amount of ethanol (EtOH) consumed in g/kg at various doses of naltrexone. Based on the number of lever press responses following a dose of naltrexone (FIG. 1A), the corresponding amount of absolute ethanol consumed (g/kg) was calculated. To calculate the amount of absolute ethanol consumed, divide the number of lever presses by 2 to obtain the number of earned reinforcements. From this number, the total volume of the alcohol “cocktail” (10% EtOH in 0.04% saccharine solution) consumed was obtained. The grams of absolute EtOH consumed=the total “cocktail” volume consumed×10% EtOH×0.793 g EtOH/mL. The amount of ethanol (g) divided by the animal's weight (kg) gives the amount of absolute ethanol consumed in g/kg.

FIG. 2 is a bar graph showing that naltrexone-induced (0.5mg/kg, sc) decrease in drinking is specific for ethanol since these animals will lever press for saccharine (following naltrexone (NTX)) to the same degree as under the non-drug baseline condition with the EtOH cocktail.

FIG. 3 is a bar graph showing increasing dosages of aripiprazole (APZ) in combination with naltrexone versus alcohol consumption as indicated by the number of lever presses.

FIG. 4 is a bar graph showing that coadministration of naltrexone does not affect aripiprazole's anticonvulsant activities (as measured by the withdrawal rating).

### DETAILED DESCRIPTION

As stated in Swift, R. supra at 1488,
Although therapy with two or more drugs with different mechanisms of action, given together or in sequence, may yield additive or synergistic benefits in patients with alcohol dependence, there is no evidence that multiple-drug therapy improves the effectiveness of treatment.
   Applicant has analyzed various drug combinations and has identified combinations of drugs which are particularly suitable for the treatment of alcoholism, including abatement of adverse clinical manifestations of treatment which affect patient compliance and hence, the overall success of treatment. The method of the administering combinations of selected drugs enables full spectrum treatment from detoxification through rehabilitation. A particular advantage of the invention is that it provides a method of continual support for the recovering alcoholic beyond rehabilitation. The patient has the psychological advantage of having alternatives in times of weakness beyond classical treatment when active support is discontinued, for example, discontinuation of individual or group therapy, residential treatment in alcohol-free settings and self-help groups. That is, the combination treatment of the invention can be continued or renewed as medically indicated before (in anticipation of) or concurrently with life events that would increase the risk of relapse.

When assessing success of drug treatments, typical outcomes include, but are not limited to, increases in abstinence, expressed as the proportion of patients remaining abstinent or the length of time to the loss of abstinence (relapse), and reductions in the quantity or frequency of drinking, expressed as the number of drinking days and the number of drinks per drinking day. Although abstinence is the more stringent outcome and is preferred, reductions in consumption can nevertheless reduce alcohol-related morbidity. Further, retention of the patient in the treatment regime is an important indicator of success. A high drop-out rate of patients on naltrexone alone compared to those with the combination therapy of the invention can be easily determined.

In one embodiment, the current invention combines the use of a dopamine D2 partial agonist with an opioid antagonist for the treatment of alcoholism. Unless otherwise indicated, as used herein the treatment of alcoholism includes the treatment of alcohol dependence, withdrawal symptoms, PAS and cravings.

As used herein “treatment” of alcoholism includes the treatment of initial and ongoing symptoms of alcoholism, prophylactic treatment of patients susceptible to relapse of alcoholism, treatment of patients who have relapsed into alcoholism. As used herein a “susceptible” patient is a patient that has the potential of having a relapse of disease for any reason including times of weakness beyond classical treatment when active support is discontinued, for example, discontinuation of individual or group therapy, residential treatment in alcohol-free settings and self-help groups or any other life events that would increase the risk of relapse.

As used herein the term “inhibiting the undesirable adverse clinical manifestations of alcoholism” refers to preventing, partially or totally, symptoms often associated with treatment for alcoholism including but not limited to (generally in order of increasing severity): feelings of jumpiness or nervousness; feeling of shakiness; anxiety; irritability or being easily excited; difficulty in thinking clearly; bad dreams; emotional volatility; rapid emotional changes; depression; fatigue; headache (generally pulsating); sweating (especially palms of the hands or the face); nausea; vomiting; loss of appetite; insomnia or sleeping difficulty; paleness; rapid heart rate (palpitations); eyes, especially pupils, different size (enlarged, dilated pupils); clammy skin; abnormal movements including tremor of the hands or involuntary, abnormal movements of the eyelids; state of confusion and hallucinations (also called delirium tremens); agitation; fever; convulsions; “black outs”. (Source: National Institutes of Health) By combining the actions of two or more drugs of the invention, an alcoholic patient's symptoms and cravings will be treated at the same time. In one embodiment, two different types of drugs, a patient's symptoms and cravings will be treated at the same time. The two different types of drugs reduce neuronal hyperexcitability associated with withdrawal or PAS while, at the same time, blocking the craving for or the positive reinforcing effects of alcohol. The drug combination would also likely help reinforce the efficacy of each drug in a number of ways. In reducing withdrawal symptoms, dopamine D2 partial agonist help reduce the craving for alcohol that accompanies withdrawal. Since insomnia is a common symptom of alcohol-dependent patients, the sleep inducing properties of partial D2 agonists are also beneficial to patients. This further reduces withdrawal symptoms and further reinforces the actions of the opioid antagonist. The drug combination would significantly reduce the likelihood of relapse as well as help increase compliance and successful treatment outcomes. All treatments are not successful. However, by removing “non-compliance” with the treatment regime as a factor, reasons for failure of treatment come into better focus and allow interventions which are more tailored to the patient.

Combined treatment of a dopamine D2 partial agonist and the opioid antagonist would continue through and until withdrawal and/or PAS symptoms abated. Also, combined treatment would continue throughout an abstinence or drinking reduction program or be administered in anticipation of, or concurrently with, life events that would increase the risk of relapse. Initial combined treatment of the dopamine D2 partial agonist and the opioid antagonist would continue from a period of from one month to about six months. Suitable doses of dopamine D2 partial agonists are at low enough doses to lower or reduce the undesirable adverse clinical manifestations while still eliciting the reinforcing or positive effect with the opioid antagonist. The preferred time of day for administering the dose would be the evening or before bedtime.

In one embodiment the naltrexone is naltrexone hydrochloride (HCl) which is available generically and under the trade name ReVia® or Depade®. Naltrexone is currently available in oral tablet from and is approved by the U.S. Food and Drug Administration (FDA) for the treatment of alcoholism as well as heroin and opium addiction. While not being held to one particular theory, it is believe that opioid antagonists act by blocking the positive reinforcing effect of alcohol, which results from the release of endogenous opioids upon the consumption of alcohol. In general, opioid antagonists are used in the treatment of alcoholism following a period of abstinence by the patient, which may include symptoms of withdrawal. Most patients take naltrexone for 12 weeks or more. In general, the treatment involves taking a prescribed course of naltrexone tablets for up to one year. These tablets are taken by mouth, once a day or, every couple of days at a higher dose. Generally, the doctor may initially monitor the patient's progress quite closely. Naltrexone's effects on blocking opioids occur shortly after taking the first dose. Findings to date suggest that the effects of naltrexone in helping patients remain abstinent and avoid relapse to alcohol use also occur early.

Naltrexone is dispensed by retail or mail-order pharmacies. Taking naltrexone tablets is only part of the treatment. As in many other conditions, the treatment can be more effective when combined with counseling and ongoing support from friends and family. It appears that patients who do have the involvement of a caregiver are more likely to complete the naltrexone treatment. For this reason doctors may encourage the patient to seek out people they can rely on for support and care during the treatment. This could include a family member, a partner, friend or a health practitioner such as nurse or pharmacist. One of the key roles for the caregiver is to supervise the naltrexone dosage as prescribed by the doctor. Even with the support of the caregiver, the treatment can be jeopardized by a potential for conflict which may arise as some patient may come to resent being supervised. Further, some patients do not have access to a caregiver. These patients are especially at risk for unsuccessful treatment.

It is know that some patients have adverse clinical manifestations like nausea, headache, constipation, dizziness, nervousness, insomnia, drowsiness, anxiety and other symptoms disclosed above. Naltrexone adverse clinical manifestations, predominantly nausea, have been severe enough to discontinue the medication in 5-10% of the patients prescribed it as a treatment for alcoholism. If a patient gets any of these adverse clinical manifestations and consults the doctor, the doctor may be forced to change the treatment or suggest other ways to deal with the adverse clinical manifestations. Often instead of seeing a doctor, the patient will “self-treat” by skipping doses or stopping the doses altogether.

Combination of Opioid Antagonists and Dopamine D2 Partial Agonist

Blockade of the D2 dopamine receptors, inhibiting dopamine-mediated neurotransmission, produces an antipsychotic response. Anti-dopaminergic drug treatments are effective in reducing psychotic symptoms of schizophrenia (i.e. positive symptoms) but leave other aspects of the illness (e.g. negative symptoms, attention, concentration, and cognition) poorly treated. Dopamine receptor partial agonists, i.e. dopamine receptor ligands with some but not full intrinsic activity at dopamine receptors, are being developed to achieve an optimal degree of stimulation at dopamine receptors, avoiding excessive dopamine receptor blockade or excessive stimulation. Such partial agonists would act as dopamine system stabilizers without motor side effects (see Stahl S M, “Dopamine system stabilizers, aripiprazole, and the next generation of antipsychotics, part 1, “Goldilocks” actions at dopamine receptors,” J Clin Psychiatry November 2001; 62(11):841-2).

Aripiprazole is an example of a dopamine D2 partial agonist. Aripiprazole, 7-{4-[4-(2,3-dichlorophenyl)-1-piperazinyl]-butoxy}-3-,4-dihydro carbostyril or 7-{4-(2,3-dichlorophenyl)-1-piperazinyl]-butoxy}-3,4-dihydro-2(1H)-quinolinone, is an atypical antipsychotic agent useful for the treatment of schizophrenia (U.S. Pat. No. 4,734,416 and U.S. Pat. No. 5,006,528).

Other examples include the dopamine partial agonist (−)3-(3-hydroxyphenyl)-N-n-propylpiperidine [(−)-3PPP, see U.S. Pat. No. 4,719,219]. The drug has several distinctive properties in clinical application in schizophrenia. The antipsychotic action of (−)-3PPP is broad, including both positive and negative symptoms, and is not accompanied by any acute motor side effect, no Parkinsonian symptoms, no akathisia, no dysphoria.

### EXEMPLIFICATION

### Example 1

The objective of this study is to determine whether naltrexone's ability to decrease alcohol consumption is affected when combined with a dopamine D2 partial agonist. The model used for this study is a rat model of alcohol self-administration.

Methods

Animals

Male Wistar rats (starting weight of 200±30 grams; Charles River Laboratories, Mass.) were individually housed with free access to food and water. The vivarium was maintained within the temperature and relative humidity range specified within the Guide for Care and Use of Laboratory Animals (NIH Publication No. 86-23, revised 1985). These conditions were recorded once daily throughout the study. The vivarium was on a 12 hour light/dark schedule. All animal studies were reviewed and approved by the Alkermes' IACUC (protocol #04-2A).

Ethanol Self Administration Training Procedure

Animals were trained daily in an operant chamber to press a lever to receive access to an ethanol cocktail as a reinforcer using a saccharin fading procedure. This procedure began with a highly sweetened saccharin solution (0.1%) and increasing amounts of ethanol were gradually introduced over a period of 2-3 weeks while the sweetness was continually reduced. The final ethanol cocktail contained 10% ethanol in 0.04% saccharine. Each session lasted 30 minutes, during which the rat could press the lever twice to gain access to 0.1 mL of the ethanol cocktail. The operant chamber (Coulbourne Instruments, Allentown, Pa.) is a computer-controlled automated system which recorded the number of lever presses completed by a rat. At the end of the training period (6-8 weeks), rats which consistently drank a sufficient quantity of ethanol to produce a pharmacological effect (minimum intake of 0.6 g/kg/hour) were selected to participate in the drug studies. These trained rats were used repeatedly throughout these studies to control for intra-subject variability. All drugs were administered acutely with a minimum of a 2 day drug washout period.

Drug Preparation

Naltrexone was prepared daily in 0.9% saline and administered subcutaneously (SC). The dopamine D2 partial agonist aripiprazole was suspended in 3% carboxymethyl cellulose; a total volume of 1 mL/kg of this suspension was delivered orally (PO) to the rat using a gavage tube. See Table 1 for source and lot numbers of the drugs tested.

TABLE 1
Drug Information

| DRUG | SOURCE | LOT NUMBER |
|---|---|---|
| Naltrexone | Sigma, Inc. | 103K1495 |
| Aripiprazole | Dr. Reddy's | APZ-4 001 04 |
| | Laboratories, LTD. | |

Effect of Naltrexone on Ethanol Drinking

The ability of naltrexone to reduce ethanol drinking (i.e., decrease the number of lever presses) was assessed in this animal model of self administration of ethanol. Thirty minutes after the administration of naltrexone (0-6 mg/kg, SC), the animals were placed in the operant chamber and allowed to lever press for the 10% ethanol cocktail. The total number of lever presses was recorded over the 30 minute test session. The rats were repeatedly dosed with naltrexone to generate a dose-response curve for each individual animal. To determine if naltrexone specifically decreased ethanol drinking (as opposed to drinking in general), a 0.1% saccharine solution was substituted for the ethanol cocktail.

Effect of the Coadministration of a Dopamine D2 Partial Agonist with Naltrexone on Ethanol Drinking

The dopamine D2 partial agonist aripiprazole was coadministered with naltrexone to determine if it affected naltrexone's ability to decease ethanol drinking. The dose of naltrexone used in this series of studies was the ED₅₀ (that is, the dose of naltrexone that produced a 50% decrease in lever responses for ethanol as determined from the dose-response study). This dose allows one to determine if the coadministered drugs impaired or enhanced naltrexone's effect on ethanol drinking. Aripiprazole was administered orally 30 minutes prior to a naltrexone injection (SC) (i.e., 60 minutes prior to the beginning of the ethanol drinking test session). The number of lever presses for the ethanol cocktail was recorded at the end of the 30 minute session.

Results

Effect of Naltrexone on Ethanol Drinking

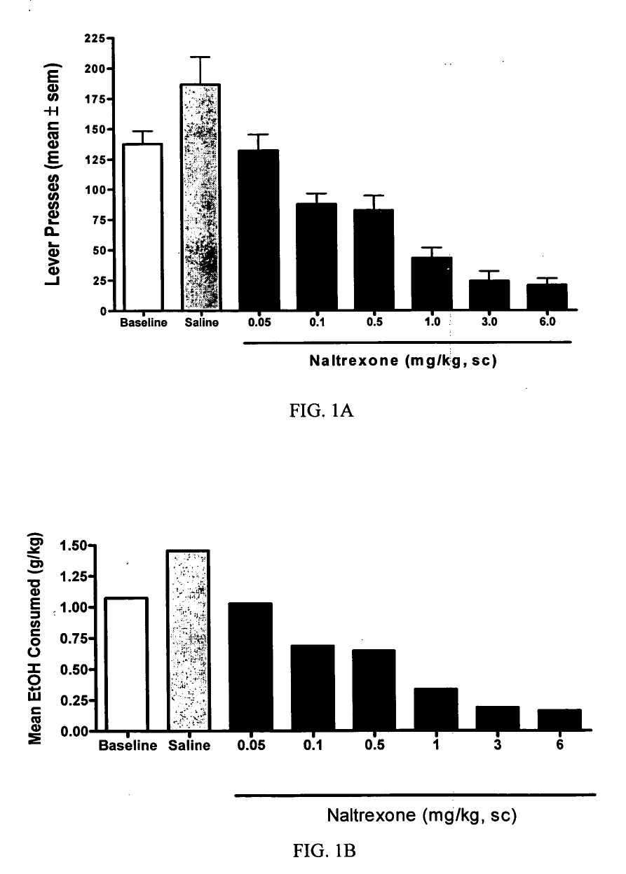

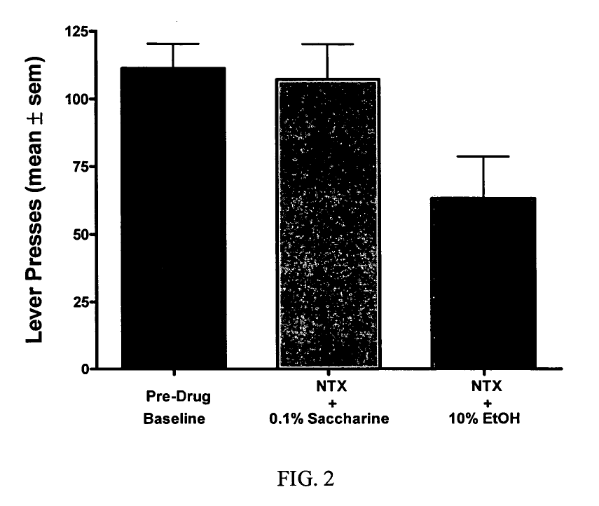

Efficacy of naltrexone was confirmed in the behavioral model of ethanol self administration, as indicated by a dose-dependent decrease in the number of lever presses by treated rats (Table 2, FIG. 1A). In contrast, there was no significant decrease between the baseline (no drug treatment), vehicle control (saline) and the lowest dose of naltrexone tested (0.05 mg/kg). At the higher doses (3 and 6 mg/kg), the effect of naltrexone on decreasing ethanol drinking appeared to plateau (bottom out). The naltrexone ED₅₀ was determined to be 0.5 mg/kg, which was defined as the dose at which responding to the lever for ethanol was reduced by approximately 50% compared to baseline values. Similarly, the amount of absolute ethanol consumed (g/kg) following naltrexone administration also confirms the ED₅₀ dose of 0.5 mg/kg as being 50% less than the baseline (FIG. 1B). Additionally, naltrexone was shown at this dose to be selective for decreasing ethanol drinking in rats (but not saccharine drinking) (FIG. 2).

TABLE 2
Naltrexone Dose-Response

| | | | | Approximate |
|---|---|---|---|---|
| | | | | Absolute Ethanol |
| | Dose | | Lever Presses | Consumed |
| Treatment | (mg/kg) | N | (Mean ± SEM) | (g/kg) |
| No Drug | — | 9 | 138 ± 10.6 | 1.1 |
| (Baseline) | | | | |
| Naltrexone | Vehicle | 7 | 187 ± 22.75 | 1.5 |
| | 0.05 | 9 | 132 ± 13.3 | 1.0 |
| | 0.1 | 9 | 88 ± 8.6 | 0.7 |
| | 0.5 | 8 | 83 ± 12.1 | 0.6 |
| | 1.0 | 7 | 43 ± 8.6 | 0.3 |
| | 3.0 | 6 | 24 ± 8.1 | 0.2 |
| | 6.0 | 6 | 21 ± 5.5 | 0.2 |

Effect of the Coadministration of a Dopamine D2 Partial Agonist with Naltrexone on Ethanol Drinking

This phase of the study investigated the effect of potential drug interactions between naltrexone and aripiprazole on the number of lever presses by rats for ethanol compared to naltrexone alone. A significantly higher number of lever responses would demonstrate that the drug interaction impaired naltrexone's ability to decrease ethanol drinking. In contrast, significantly lower responses would suggest a synergistic or additive effect of the drug combination (Table 3).

TABLE 3
Drug Interaction Studies

| | | ED₅₀ | |
|---|---|---|---|
| TREATMENT | DRUG DOSE | NALTREXONE | |
| (TRADE NAME) | (PO, MG/KG) | (SC, MG/KG) | N |
| ARIPIPRAZOLE | 0.0 | 0.1 | 15 |
| (Abilify ®) | 0.1 | | 13 |
| | 1.0 | | 14 |
| | 10.0 | | 12 |

Aripiprazole

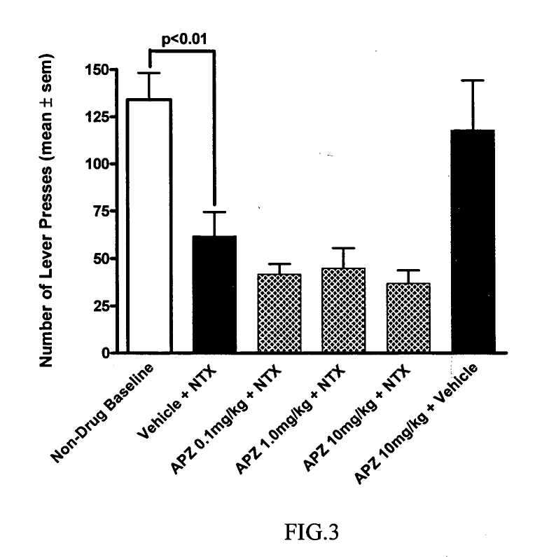

Aripiprazole (Abilify®), a dopamine D₂ partial agonist, is an antipsychotic commonly used in the treatment for schizophrenia and the mania phase of bipolar disorder (manic-depressive illness). It was also tested with naltrexone to evaluate its effects on ethanol drinking. A dose range of 0.1 mg/kg-10 mg/kg (PO) of aripiprazole was tested. The naltrexone alone group showed a 47.54% decrease in the number of lever presses, when compared to baseline. There was no statistical difference in the number of ethanol-motivated lever presses as a result of the combination of naltrexone and aripiprazole versus naltrexone alone (FIG. 3, Table 4). Thus, at the doses tested, there was no drug interaction between naltrexone and aripiprazole.

TABLE 4
Coadministration of Aripiprazole Does Not Affect Naltrexone's Ability to
Decrease Alcohol Drinking

| | | | | Number of |
|---|---|---|---|---|
| | Drug Dose | ED₅₀ Naltrexone | | Lever Presses |
| Treatment | (PO, mg/kg) | (SC, mg/kg) | N | (mean ± sem) |
| Aripiprazole | — | Non-Drug Baseline | 15 | 134 ± 14 |
| | 0.0 | 0.1 | 15 | 62 ± 13 |
| | 0.1 | | 13 | 42 ± 11 |
| | 1.0 | | 15 | 45 ± 8 |
| | 10.0 | | 12 | 37 ± 7 |
| | 10.0 | — | 7 | 118 ± 26 |

Compared to vehicle+naltrexone treatment (bolded values), coadministration of aripiprazole does not affect (i.e., neither impairs nor enhances) naltrexone's ability to suppress the self administration of EtOH in rats.
Conclusion

When coadministered with naltrexone, the dopamine D² partial agonist aripiprazole did not impair or block naltrexone's ability to decrease drinking in a rat model of alcohol self-administration. These data support the use of the coadministration of dopamine D2 partial agonists with opioid antagonists for the treatment of alcohol dependency and symptoms associated with alcohol withdrawal.

### Example 2

A dopamine partial D2 agonist (aripiprazole) was studied in a mouse model of alcohol withdrawal. Possible interactions with aripiprazole's ability to reduce convulsions when combined with naltrexone was also studied.

Methods

Animals

Male C57BL/6 mice (starting weight of 15-18 grams; Charles River Laboratories, Mass.) were housed in groups of 4 on a ventilated rack with free access to food and water. The vivarium was maintained within the temperature and relative humidity range specified within the Guide for Care and Use of Laboratory Animals (NIH Publication No. 86-23, revised 1985). These conditions were recorded once daily throughout the study. The vivarium was on a 12 hour light/dark schedule. All animal studies were reviewed and approved by the Alkermes' IACUC (protocol #04-8A).

Alcohol Administration for the Induction of Alcohol Withdrawal Symptoms

Persistent high alcohol levels in blood and brain are required to develop withdrawal symptoms following termination of alcohol availability. Because rodents do not consistently consume large amounts of alcohol voluntarily, one approach that has been used is “forced” choice administration. Alcohol is provided in a nutritionally balanced liquid diet. This diet becomes the animal's sole source of food and water. While the experimenter controls the duration of the alcohol exposure, the animal determines the dose and pattern of consumption.

After a 3-5 day acclimation period, the mice were individually identified (tail mark with permanent marker) and weighed. The normal rodent chow and water was replaced with a nutritionally complete control liquid diet (Bio-Serv, Lieber-DeCarli diet) for a 5-7 day habituation period. Acquisition of EtOH drinking in mice involved a gradually escalating ethanol concentration procedure. Ethanol treated animals received an EtOH liquid diet containing 1.5% EtOH for 5-7 days. The EtOH concentration was increased every 5-7 days to a final concentration of 6.7%. The liquid diet was administered in a screw capped graduated 100 mL liquid feeding tube mounted inside the cage. The volume of remaining diet was measured and the diet changed daily. The mice were observed daily and weighed every week to assure adequate EtOH diet intake.

Development of alcohol withdrawal symptoms requires a cycle of alcohol/no alcohol. The animals were given a period of 14 days of the EtOH diet (6.7%), 2 days of control diet (balanced isocalorically with maltose dextrin replacing the EtOH) and 5-6 days of the EtOH diet.

Audiogenic—Induced Convulsions

One of the hallmark symptoms of alcohol withdrawal in mice is the occurrence of convulsions. These can occur spontaneously, from handling the animal, from a sudden loud sound (audiogenic) or from subthreshold doses of chemical convulsants. For this series of studies, the occurrence of audiogenic-induced convulsions was used as a measure of alcohol withdrawal. To test for audiogenic-induced convulsions, each group (cage) of mice was placed in a clear 5 gallon polystyrene bucket with a thin layer of corn cob bedding on its floor. After a 3 minute acclimation period (quiet period), a 78 db electric bell, located 3 feet above the bucket, was activated for 30 seconds and the animals' behavior was observed. A 4-point scale of the behavioral response was developed and used, where 1=freezing response, 2=hyper-reactive response involving jumping and running, 3=tonic-clonic convulsions with survival, and 4=tonic-clonic convulsions followed by death.

Drug Preparation

Naltrexone (Sigma, Inc., Lot # 103K1495) was prepared daily for a dose of 3 mg/kg in 0.9% saline and administered intraperitioneally (IP). The dopamine D2 partial agonist aripiprazole (Dr. Reddy's Laboratories, LTD, Lot# APZ-4 001 04) was suspended in 3% carboxymethyl cellulose for a dose of 10 mg/kg; a total volume of 1 mL/100 g of this suspension was delivered orally (PO) to the mouse using a gavage tube.

Effect of Naltrexone on Aripiprazole's Anticonvulsant Activity

Mice from each cage were randomly assigned to one of three groups, vehicle+saline, aripiprazole (10 mg/kg, PO)+saline, or aripiprazole (10 mg/kg, PO)+naltrexone (3 mg/kg, IP). The aripiprazole or vehicle was dosed 60 minutes before testing and followed by a naltrexone or saline injection 30 minutes later. The mice were isolated from the test room and bell sound prior to testing. Each mouse's behavior in response to the bell was observed by two investigators and the agreed upon results were recorded.

Results

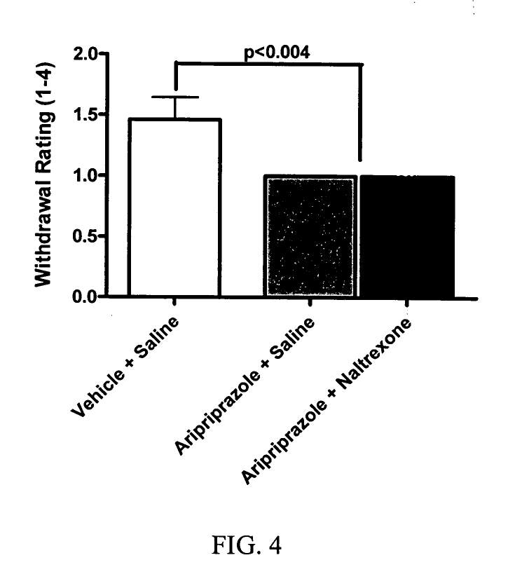

Prior to testing, several mice were observed to exhibited spontaneous convulsions and several were found dead following the removal of the EtOH diet. Mice also appeared hyperactive with increased vocalizations when handled. While there was a significant drug effect (anticonvulsant effect) of aripiprazole with or without naltrexone, no statistical differences were found between the naltrexone and non-naltrexone groups on the audiogenic convulsion test (p=0.004, FIG. 4, Table 5). The data do indicate that naltrexone did not interact with aripiprazole at the dose tested and did not exacerbate the audiogenic-induced convulsions in mice exhibiting symptoms of alcohol withdrawal.

TABLE 5
Coadministration of Aripiprazole and Naltrexone on Audiogenic-Induced
Convulsions

| Aripiprazole | Naltrexone | | Withdrawal Rating* |
|---|---|---|---|
| (10 mg/kg, PO) | (3 mg/kg, SC) | N | (mean ± sem) |
| Vehicle | Saline | 13 | 1.5 ± 0.2 |
| Aripiprazole | Saline | 12 | 1.0 ± 0.0 |
| Aripiprazole | Naltrexone | 14 | 1.0 ± 0.0 |

*Behavioral response using a scale of 1-4 (see text)

Conclusions

In an animal model of alcohol withdrawal, naltrexone did not appear to interact (block or enhance) aripiprazole's anticonvulsant activity on an audiogenic-induced convulsion test.

### Example 3

The objective of this study is to determine whether opioid antagonists in combination with dopamine D2 partial agonist increase the compliance as a treatment regime compared to treatment using the opioid antagonist alone. This endpoint of increased compliance is significant whether or not the treatment regime itself is successful, that is decreasing or eliminating alcohol consumption. It is important for the treating physician to know whether treatment failed because of “non-compliance” versus lack of responsiveness to the drug of choice or the dose of that drug, for example.

Patients are observed initially in an in-patient setting of a hospital (for detoxification) and a community clinic (for follow-up). Alcoholics seeking withdrawal are selected for the study. Enrollment is conducted as follows: Enrollment criteria is current dependence and wish to transfer to naltrexone maintenance. Exclusion criteria include serious psychiatric problems, serious medical problems, especially acute liver disease or kidney damage, pregnancy, and concurrent drug addiction, especially benzodiazepine or heroin dependence.

Upon enrollment, all patients are interviewed by a nurse and a doctor. Interviews last about an hour and cover alcohol use, including any drug use, treatment history, explanation of the proposed treatment, and exploration of patient goals and motivation. Standardized questionnaires (Severity of Dependence Scale [SDS], Severity of Alcohol Withdrawal Scale, Quality of Life Inventory and System Checklist-90, a global checklist of psychological functioning) are administered.

Each patient is allocated a case manager who attends detoxification and conduct follow-up. Case managers comprise a psychologist, a registered nurse and a pharmacist with counseling qualifications.

The initial detoxification uses opioid antagonists either alone or in combination with a dopamine D2 partial agonist. Patients are discharged when they feel well enough. Follow-up is daily for four days and then weekly for up to three months for supportive care.

The main outcome measurements include (A) the severity of adverse clinical manifestations; patient ratings of severity and acceptability of withdrawal; nights of hospitalization; rates of induction onto naltrexone; retention in treatment over three months; and relapse to alcohol use and (B) given success or failure of treatment based upon (A) above, did the patient receive (1) a combined dose of the naltrexone and the dopamine D2 partial agonist of the invention in a single combination pill, (2) the combined dose of the instant invention in two different pills (one for naltrexone and one for the dopamine D2 partial agonist) or (3) naltrexone alone.

Modifications and variations of the invention will be obvious to those skilled in the art from the foregoing detailed description of the invention. Such modifications and variations are intended to come within the scope of the appended claims.

1. A method for treating alcoholism comprising administering to a patient a therapeutically effective amount of a combination of:
(i) at least one opioid antagonist; and
(ii) at least one dopamine D2 partial agonist.

2. The method of claim 1 wherein the at least one dopamine D2 partial agonist reduces the negative adverse clinical manifestations of the at least one opioid antagonist.

3. The method of claim 1 wherein the at least one opioid antagonist is selected from the group consisting of naltrexone, naloxone and nalmefene.

4. The method of claim 1 wherein said at least one opioid antagonist is in the form selected from the group consisting of a polymorph, solvate, hydrate, dehydrate, co-crystal, anhydrous form and amorphous form or combinations thereof.

5. The method of claim 4 wherein said at least one dopamine D2 partial agonist is selected from the group consisting of Aripiprazole and (−)3-(3-hydroxyphenyl)-N-n-propylpiperidine [(−)-3PPP].

6. The method of claim 5 wherein the composition reduces the adverse clinical manifestations of said at least one opioid antagonist as compared to the adverse clinical manifestations from the administration of the same level of said at least one opioid antagonist alone.

7. A method according to claim 5 wherein said administration is selected from the group consisting of oral administered, intravenous injection, intramuscular injection, intradermal injection, a depot version of intradermal administration, implants, parenteral administration and combinations of these.

8. A treatment regimen for treating alcoholism in a patient comprising administering simultaneously or sequentially, as a single treatment episode, a combination of at least one opioid antagonist and at least one dopamine D2 partial agonist.

9. A treatment regimen for inhibiting the relapse of alcoholism in a susceptible patient comprising administering simultaneously or sequentially, as a single treatment episode, a combination of at least one opioid antagonist and at least one dopamine D2 partial agonist.

10. A method for inhibiting the relapse of alcoholism in a susceptible patient comprising administering to the patient a composition comprising a combination of at least one opioid antagonist and at least one dopamine D2 partial agonist in an amount sufficient to inhibit the relapse of alcoholism.

11. A method of inhibiting the onset of undesirable adverse clinical manifestations associated with the treatment of alcoholism comprising administering simultaneously or sequentially, as a single treatment episode, a combination of at least one opioid antagonist and at least one dopamine D2 partial agonist.

12. A method of enhancing patient compliance of a patient undergoing treatment for alcoholism comprising administering simultaneously or sequentially, as a single treatment episode, a combination of at least one opioid antagonist and at least one dopamine D2 partial agonist.

13. A method of enhancing patient compliance of a patient undergoing treatment for alcoholism comprising administering a composition comprising a combination of at least one opioid antagonist and at least one dopamine D2 partial agonist in a therapeutically effective amount.

14. A kit comprising at least one treatment dose of therapeutically effective amount of:
(i) at least one opioid antagonist; and
(ii) at least one dopamine D2 partial agonist
wherein the antagonist of (i) and the dopamine D2 partial agonist of (ii) comprise a single pharmaceutical composition.

15. A kit comprising at least one treatment dose of therapeutically effective amounts of:
(i) at least one opioid antagonist; and
(ii) at least one dopamine D2 partial agonist
wherein the antagonist of (i) and the dopamine D2 partial agonist of (ii) comprise more than one pharmaceutical composition.

16. The kit of claim 14 wherein the single pharmaceutical composition is a form of oral medication.

17. The kit of claim 16 wherein the pharmaceutical composition is in the form selected from the group consisting of a pill, capsule, wafer, tablet and caplet.

18. The kit of claim 15 wherein the more than one pharmaceutical composition is an oral medication.

19. The kit of claim 18 wherein the more than one pharmaceutical composition is in the form selected from the group consisting of a pill, capsule, wafer, tablet, caplet or any combination thereof.
